(19) United States
(12) Patent Application Publication
(10) Pub. No.: US 2006/0210654 A1
(43) Pub. Date: Sep. 21, 2006

(54) Process for extracting vital components from plants

(75) Inventor: Refael Aharon, Modiin (IL)

Correspondence Address: WILLIAM J. SAPONE;COLEMAN SUDOL SAPONE P.C., 714 COLORADO AVENUE, BRIDGE PORT, CT, 06605, US

(73) Assignee: Green Plant-Based Industries, LTD., Modiin (IL)

(21) Appl. No.: 10/566,517

(22) PCT Filed: Aug. 4, 2004
(86) PCT No.: PCT/IL04/00725

§ 371 Date: Jan. 30, 2006

(30) Foreign Application Priority Data
Aug. 4, 2003 (IL) 157233

(51) Int. Cl. A61K 36/53; A61K 36/537; A61K 36/534; A23C 1/06

(52) U.S. Cl. 424/745; 424/746; 424/747; 426/384

(57) ABSTRACT

The present invention presents a cost effective process for separating between liquids extracted from whole plants or plant parts and the solid plant residue, and performing concurrent sterilization of both substances. This novel method comprising fast deep-freezing said whole plant(s) or parts thereof; crushing the deep-frozen whole plant(s), or parts thereof, thereby obtaining a bioextract; defrosting said bioextract mixture so as to allow drainage of the liquids from the defrosted bioextract mixture; and, separating between the liquids and the solid substance of the defrosted bioextract mixture such that a sterile product is obtained. The present invention also discloses sterile cosmetic, food, beverage, nutraceutical or pharmaceutical compositions, which comprises a natural mineral-rich plant tissue(s) component(s) obtained by this process.

### FILED OF THE INVENTION

The present invention generally relates to the field of cost effective production of sterile cosmetics, foods, beverages, nutraceutical and pharmaceutical compositions.

### BACKGROUND OF THE INVENTION

Well-known technologies for extracting useful components from plants exist and are exploited on a daily basis. For example, substances from plants belonging to the labiatae family have been extracted to provide antioxidants, e.g., U.S. Pat. No. 6,383,543 to Reznik. Other substances extracted from different plants, are exploited in anti-inflammatory, antiallergic and cosmetic applications, e.g., U.S. Pat. No. 6,060,061 to Breton et al. It is also well known that essential oils extracted from plants of the labiatae family are useful as food flavorings, e.g., U.S. Pat. No. 6,306,450 to Bank et al. The contents of the aforementioned U.S. patents, including references cited therein, are incorporated herein by reference. Medical virtues of plants belonging to the labiatae family are known from scientific literature and may be exemplified by those mentioned in the above-cited U.S. Pat. Nos. 6,383,543 and 6,060,061.

It will be appreciated by those skilled in the art that useful substances, such as those mentioned above, exist in plant extracts, where the extraction has been carried out example by use of water or organic solvents. In such extraction processes, the insoluble plant residues loose a considerable portion of their original healthy secondary metabolite characteristics, and, therefore, are generally believed to have little value and are discarded, or at most used for animal feed.

### SUMMARY OF THE INVENTION

The present inventors have surprisingly found that such generally discarded plant residues, which remain after removal of the liquid phase extracts, are themselves of cosmetic, food, nutritional, nutraceutical (i.e., a medicament or other pharmaceutical product that has nutritional value; and/or a food that has had its nutritional value-enhanced by pharmaceuticals) and pharmaceutical significance. Moreover, the separation between liquids extracted from plants, as disclosed in the present invention, together with the concurrent sterilization process, which is also disclosed in the present invention, were found to be helpful in preserving the original healthy secondary metabolite characteristics both of the extracted liquids and the solids residues.

The present invention provides hence a cost effective process for separating between, and concurrently sterilizing the liquids and solid substances of whole plants or any parts of plants. The sterilized liquids and solid products are adapted to be utilized as ingredients for cosmetic, food, beverage, nutraceutical and pharmaceutical compositions.

The term ‘bioextract’ refers hereinafter to any composition of matter obtained adjacent to mechanically crushing frozen plants, or any parts thereof. The composition of matter may be selected inter alia from a mixture, suspension, emulsion comprising liquids and solid substances of the frozen crushed plant(s). It is in the scope of the present invention wherein he process for separating between liquids and solid substances of whole plants or parts of plants and concurrently sterilizing them comprises the steps of fast deep-freezing the plant(s) or parts thereof, such as by immersing the plant(s) in liquid nitrogen, frozen fluids or other cold liquids or gases crushing the whole deep-frozen plant(s), or parts thereof; defrosting the resulting bioextract mixture so as to allow draining of the liquids from the defrosted bioextract; and separating between the liquids and the solid substances of the defrosted bioextract mixture, preferably by squeezing the solids and liquids off the bioextract by employing high pressure thereon.

In the above-mentioned crushing stage, it has been found that crushing plants, or parts thereof, which have been deep-frozen prior to the crushing stage, produces particles much smaller than those that could have been obtained by utilization of conventional crushing methods, because a deep-frozen substance tends, when a crushing force is exerted thereon, to disintegrate into many tiny pieces, similar to broken glass. As a result, more liquid can be extracted from plants, with several consequences: a given quantity of plants or parts thereof, yields more liquid, in comparison to conventional separation methods, and the resulting solid residues are therefore drier, which normally extends their lifespan. Both consequences are of considerable economical significance.

It has also been found that a preceding stage of deep-freezing of the whole plant, or parts thereof, imparts to the resulting separated liquids and solid substances a considerable degree of sterilization, as illustrated in the experiments described below. Preferably, the separated liquids are filtered and further sterilized and the solid substances, which essentially contain the original richness of mineral content, due to the freezing step in the separation process, are “cold” sterilized and homogenized by processing the solid residues to an average particle size of no more than about 0.6 micron. The (−cold) sterilization process employed on the separated liquids and solids substances, comprises one or more “freeze-defrost” cycle(s), each of which includes one fast deep-freeze of the liquid(s) and solid substances followed by one fast defrost of the frozen liquids/solids.

Preferably, in each freeze-defrost cycle the liquids/solids substances are kept frozen at a temperature within the range of about −130° C. to −197° C. for a period of about 10 seconds or until the whole tissue is subjected to the desirable temperature, after which the frozen liquids/solids are fast defrosted to a temperature within the range of about 15° C.÷20° C., by immersing them in hot liquid, such as water, whose initial temperature is between about 80° C. and 90° C.

In each cycle of the sterilization process, the liquids/solids substances are frozen by utilizing liquid nitrogen, or other suitable fluid.

The sterilized liquids and/or solids substances could be utilized as ingredients in cosmetic, food, beverage, nutraceutical and pharmaceutical compositions by mixing them with at least one intermediator, selected in a non limiting manner from carriers, diluents, solvents, immersions, water miscible or immiscible extractants or fluids, or without utilizing any intermediator, in which case the sterilized liquids and/or solids substances could be utilized as a concentrated product.

The process for separating liquids from solids substances in whole plants or parts of plants may be further characterized by at least one of the following features:
a) at least one of the plants belongs to the labiatae family;
b) at least one of the plants is selected from the group of: Lavandula, Melissa, Mint, Ocimum, Origanum, Preslia, Rosmarinus, Salvia, Thymus;
c) leaves and/or shoots of the plant(s) are processed together with, or separately from, roots thereof; and,
d) the mixing stage is preceded by washing the solids substances with a solvent selected from water and organic solvents, and the washed solids are subjected to homogenization and/or dehydration by freezing.

The present invention also provides a process for making cosmetic, food, nutraceutical or pharmaceutical compositions, comprising the steps of: removing the liquids from whole plants, or parts of plants, after separating between the liquids and solids substances as described above (i.e., by freezing, crushing, etc.), and mixing the residual solids substances, in particular the cell walls, with at least one cosmetically or pharmaceutically acceptable carrier, diluent, solvent or extractant.

According to an aspect of the present invention, the separated liquids and/or solid substances are utilized for making a beverage, by mixing the liquids and/or solids with at least one intermediator, such as diluent and/or other ingredient suitable for human, or animal, consumption, or without mixing the liquids/solids with an intermediator, in which case the sterilized liquids and/or solids substances could be utilized as a concentrated product.

The present invention also provides cosmetic, food, nutraceutical or pharmaceutical composition, which contains a natural mineral-rich plant tissue(s) component(s). The tissue(s) component(s) are obtainable by: deep-freezing whole plant(s) or parts thereof; crushing the frozen whole plant(s) or parts thereof to a powder with particles size within the range of about 0.5 to 2 micron; defrosting the crushed product so as to allow drainage of liquids from the resulting bioextract; separating between said liquids and the solid substance of the bioextract; and, mixing solid residue(s) together with at least one acceptable carrier, diluent, solvent or extractant.

Preferably, at least one of the plants, which are utilized for making cosmetic, food, nutraceutical or pharmaceutical composition, belongs to the labiatae family. According to another aspect of the present invention, the composition(s) contains at least one plant that is selected from Lavandula, Melissa, Mint, Ocimum, Origanum, Preslia, Rosmarinus, Salvia and Thymus.

Preferably, the whole plant(s), or parts thereof, are frozen by utilizing liquid nitrogen or liquid oxygen.

According to yet another embodiment of the present invention, the solids and/or liquids of plants or of selected parts of plants, which are separated from one another as disclosed in the present invention, are utilized, wherever required, as natural colorants, flavors and/or aromatics.

### DETAILED DESCRIPTION OF THE INVENTION

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a process for separating between liquids extracted from whole plants or plant parts and the solid plant residue, and performing concurrent sterilization of both substances and any product produce thereof.

The term ‘secondary metabolite’ is hereinafter referring to natural products of metabolism that are not essential for normal growth, development or reproduction of the plant or organism. The term is also referring to special substances that are uniquely produced by each species of plants, which substances contain unique useful ingredients, such as vitamins, minerals, antioxidant agents, and other useful and vital compounds.

After separating between the liquids and solid substance of a plant, or of any parts thereof; i.e., by extracting the liquids from the plants, the extracted liquids and solids residues are useful in the foods industry), and in the cosmetic and medicine industries.

Plants to be processed according to the present invention may be selected in a non limiting manner from from Lavandula, Melissa, Mint, Ocimum, Origanum, Preslia, Rosmarinus, Salvia and Thymus. For the present invention, illustratively, Melissa officinalis L. (lemon balm), Mentha longifolia L. (horse mint), Ocimum basilicu L. (basil) and Salvia fruticosa (Greek sage) e.g. Salvia fruticosa Miller are preferred. The useful ingredients of the plant solids' residues, following separation of the liquids constituents of the plants (including solid ingredients which dissolve in the liquid phase), are health-promoting organic elements (in combined form) such as calcium, magnesium, potassium, iron, phosphorus and sulfur, while the green parts of the plants contain chlorophyll and the solid residues contain membrane proteins integrated into plant cells membranes.

In general, cosmetic compositions contain beauty-enhancing compositions as well as skin protective compositions that contain plant tissues of green leaves and/or shoots, thus plant residue, which comprises (besides other possible components) antioxidant(s) and chlorophyll is suitable for this application.

The nutraceutical and pharmaceutical compositions may be formulated in unit dosages, as is well known in the art, in which case the adjuvant ingredients may include (but are of course not limited to) a coating such as an enteric coating. The nutraceutical and pharmaceutical compositions may be formulated for, e.g., oral, dental, parenteral, rectal, topical or transdermal administration.

Foods compositions of the invention could include, for example, juice bioextract, beverages in general, and yogurts, to which extracted liquid(s) and/or solids and/or all plant tissues could be added. The actual final composition would depend on the required results. For example, Melissa may be useful in improving sleep and memory, as well as in relief of Alzheimer's disease.

It should be noted that, depending on the components and properties desired in the final product, the process for making a cosmetic, food, nutraceutical or pharmaceutical composition, could include processing leaves and/or shoots of plant(s) separately from the roots, or, alternatively the plant could be processed as a whole.

It is common practice to use liquids extracted from various plants as flavoring and coloring agents for foods and beverages, thus the present process enables economic utilization of the whole plant, i.e., by extracting more liquids from plants, in comparison to conventional methods, and by maintaining most of the original mineral richness of the various ingredients of the plants.

According to the preferred embodiment of the present invention, the extraction of liquids from a plant, or from parts thereof, is performed by: freezing the plant, or parts thereof, e.g. by use of liquid nitrogen; crushing the frozen plant, or parts thereof, under super atmospheric pressure; allowing the crushed frozen plant, or parts thereof, to warm up to a temperature, preferably no more than 4° C., at which draining or filtering the liquids is practicable; and, draining or filtering the separated liquids from the solids residues.

It is according to one embodiment of the present invention wherein Before mixing the solid residues with a carrier, either water immiscible or miscible extractants, diluents, solvents etc. the residual solids are washed with a suitable solvent, such as water miscible or organic solvents, emulsion etc. and the washed solids are subsequently homogenized and/or freeze-dried.

It is thus in the scope of the present invention wherein the solid residues are homogenized such that solid residues of an average particle size of no more than about 0.6 micron are obtained. Nevertheless, it is also in the scope of the invention wherein the aforesaid solid residues are of respectively larger average particle size of no more than about 0.6 micron are obtained.

The benefits of the present invention will now be illustrated by the following non-limiting examples:

### EXAMPLE 1

### Preparation of Solid Residues

100 g of mentha longifolia L. plants were picked with their roots from the soil. After cleaning the picked plants with sufficient amount of water, the roots, leaves (approx. 30 g) and shoots (approx 70 g) were separated from each other, after which they were frozen, by using liquid nitrogen, crushed and heated to 18° C. The thus-treated plants were then exposed to six cycles of freezing/heating, for sterilization, and the liquids were separated from the solids by employing high hydraulic pressure thereon. The extracted liquids (root and shoot liquids) were removed and the solid residues were homogenized by a homogenizer to give root and shoot solids. The analysis results of mineral content, as well as specific second metabolism components, are shown in Tables 1 and 2:

TABLE 1
Distribution of Elements (other than C, H and O)
Concentration: mg/kg dry weight (for solid); mg/l (for liquid)

| | Leaves: | Roots: | Roots: | Leaves: |
|---|---|---|---|---|
| Element | liquid | solid | liquid | solid |
| Ag | ≦0.003 | ≦0.6 | ≦0.003 | ≦0.6 |
| Al | 15 | 240 | 8.6 | 290 |
| As | ≦0.03 | ≦0.6 | ≦0.01 | ≦1 |
| B | 0.30 | 16 | 0.10 | 35 |
| Ba | 0.95 | 29 | 0.18 | 47 |
| Ca | 590 | 5100 | 55 | 15400 |
| Cd | 0.01 | ≦1 | ≦0.01 | ≦1 |
| Co | ≦0.02 | ≦2 | ≦0.02 | ≦2 |
| Cr | 0.04 | 15 | 0.08 | ≦2 |
| Cu | 0.34 | 6.5 | 0.21 | 7 |
| Fe | 12 | 280 | 6.7 | 290 |
| Hg | ≦0.01 | ≦0.4 | ≦0.01 | ≦0.4 |
| K | 970 | 4300 | 290 | 10400 |
| Li | 0.03 | ≦0.3 | 0.007 | ≦0.3 |
| Mg | 140 | 1515 | 35 | 2580 |
| Mn | 1.8 | 14 | 0.30 | 53 |
| Mo | 0.02 | ≦0.2 | 0.005 | ≦5 |
| Na | 85 | 805 | 55 | 940 |
| Ni | 0.06 | 5 | 0.04 | ≦2 |
| P | 90 | 430 | 24 | 2020 |
| Pb | ≦0.15 | ≦5 | ≦0.1 | ≦6 |
| S | 160 | 450 | 15 | 2300 |
| Se | 0.12 | ≦0.5 | 0.06 | ≦4 |
| Si | 37 | 515 | 18 | 670 |
| Sn | 0.045 | ≦0.4 | 0.035 | ≦0.5 |
| Sr | 1.9 | 46 | 0.33 | 86 |
| Ti | 0.34 | 17 | 0.20 | 24 |
| V | 0.02 | ≦0.5 | 0.012 | ≦0.5 |
| Zn | 1.8 | 21 | 0.45 | 67 |

Table 1 clearly shows that most of the useful minerals are present in the solids rather than in the liquid phase, and that there are significant differences in the mineral concentrations between the roots and the leaves.

TABLE 2
Distribution of Organic Components
Concentration: mg/kg dry weight (solid); mg/l (liquid) Concentration

| | Roots: | Roots: | Leaves: | Leaves: |
|---|---|---|---|---|
| Compound | liquid | solids | liquid | solids |
| alkene, cycloalkane, | — | 1.1 | 0.9 | 240 |
| aldehyde, polycycloalkane | | | | |
| alkane | — | 12 | <0.1 | 8300 |
| alcohols (C₈-C₁₁) | — | — | 1.8 | — |
| esters of fatty acids | — | — | <0.1 | 13 |
| Terpenes | | | | |
| limonene | — | — | — | 700 |
| α, β-phellandrene | — | — | 0.5 | 17 |
| α, β-pinene | — | — | — | 58 |
| carene | — | — | 0.1 | 1 |
| eucalyptol | — | — | 2.4 | 83 |
| terpineol | — | — | 6.2 | 48 |
| p-menth-2,8-dienol | — | — | <0.1 | — |
| (cis, trans) | | | | |
| Limonene oxide | — | — | 0.3 | 5 |
| (cis, trans) | | | | |
| borneol | — | — | 1.9 | — |
| dihydrocarveol | 0.2 | — | 8.4 | — |
| isopulegon | — | — | <0.1 | — |
| carveol | — | 1.1 | 0.3 | 16 |
| dihydrocarvone | — | — | 4.7 | 2 |
| pulegone | — | — | 0.6 | 18 |
| carvone | 0.3 | 17 | 110 | 2500 |
| carvone oxide | — | — | 0.1 | 4 |
| elemene | — | — | — | 260 |
| camphene | — | — | — | 11 |
| Sesquiterpenes | | | | |
| cubenol | — | — | <0.1 | 18 |
| caryophylene | — | 1.1 | — | 400 |
| cadinene | — | — | — | 170 |
| eremophylline | — | — | — | 2 |
| calamenene | — | — | — | 23 |
| caryophyllene oxide | — | — | — | 8 |
| cadinol | — | — | — | 14 |
| propoxyphenol | — | — | <0.1 | — |
| dimethylbenzaldehyde | — | — | <0.1 | — |
| propoxur | <0.1 | — | 0.4 | trace |
| carboximide | — | — | <0.1 | — |

Regarding table 2, most of the relative mass of the identified organic components was found in the leave solid phase. Moreover, the Gas Chromatograph Mass Spectrometer (GC-MS) apparatus is loaded with about 100,000 different secondary components.

The fact that none of the materials identified in the leaves were found in the roots indicates that the roots contain different components than the leaves.

### EXAMPLE 2

### Cosmetic Composition

A cosmetic cream was prepared in known manner using 7 g of cosmetic diluent and 2 g of solids residues that were prepared from the leaves and shoots of mentha longifolia L., as described in Example 1. The solids residues had particle size less than about 0.6 micron. An exemplary cosmetic diluent comprised magnesium lanolate 1.0%, lanoline alcohol 8.0%, paraffin oil 39.0%, methyl p-oxybenzoate 0.3% and sterile demineralized water, balance to 100%. Perfume could be conveniently added, for example, 1 ml of aqueous extract of lemon grass.

### EXAMPLE 3

### Nutraceutical Composition

1.5 g solid residues with a particle size of less than about 0.6 micron were prepared from the leaves and shoots of mentha longifolia L. as described in Example 1, and subjected to freeze-drying, after which they were mixed with a known pharmaceutical binder (i.e., microcrystalline cellulose) and filled into hard or soft gelatin capsules. Such a product could be useful as a source of essential minerals and other beneficial components.

### EXAMPLE 4

### Pharmaceutical Composition

It is known that components of the aerial parts of Rheum palaestinum have anti-platelet properties (See Phytochemistry. November 2000; 55(5):407-10). In the present example, the anti-platelet property of the Rheum palaestinum and the antioxidant activity of the labiatae were combined; by obtaining solid residues from the relevant parts in the two plants, in accordance with the process disclosed herein, and mixing them.

Method: 0.5 g of solids with a particle size of less than about 0.6 micron, were prepared from the leaves and shoots of basil as described in Example 1 and mixed with 1.5 g of solids similarly prepared from rosemary roots and with 1 g Rheum Palaestinum solids, similarly prepared from the leaves. The mixture was freeze-dried and further mixed with a known pharmaceutical binder, i.e., microcrystalline cellulose, and then filled into hard or soft gelatin capsules. The final product could potentially be exploited as an anti-platelet, relaxant and pro-digestive agent.

As mentioned above, the present invention also provides a novel sterilizing process that includes several cycles of freezing and heating the extracted liquids or solid residues. Example 5 refers to an experiment that was conducted to illustrate the advantages of the novel sterilizing process over a conventional (i.e., heat-based) sterilizing process. Example 5 also refers to the effect that the novel liquids extraction process has on the final quality/vitality of sterilized liquids, in terms of bacteriological count.

### EXAMPLE 5

### Extraction of Liquids from Fresh Mint Leaves, and Pasteurization Possibly of the Liquids

Fresh mint leaves were washed and then cleaned, with ammonium solution (0.5%), which is widely used in the salads industry for cleansing vegetables.

After the cleaning/washing stage, raw liquid was extracted or separated from the fresh leaves in two ways: (1) by utilizing a conventional method, i.e., processing the fresh leaves in a blender, and (2) by utilizing the novel extraction process, i.e., deep-freezing the leaves, crushing the frozen leaves, defrosting the leaves and extracting the liquids there from. For comparison purposes, the raw liquid obtained using the conventional method was kept and handled apart from the raw liquid obtained using the novel extraction process.

Each type of raw liquid was then filtered by manually pressing the raw liquid against thin cloth, and the filtered liquid was divided into three portions. Two samples were taken from each portion, for bacterial evaluation. The two samples taken from the first portion were sterilized in conventional manner; i.e., by immersing the extracted liquid in hot water (at approximately 80° C.) for two minutes. The two samples of the second portion were sterilized according to the novel process; i.e., the liquid went through three cycles of freezing by using liquid nitrogen and heating as described above. The two samples of the third portion were left un-sterilized, for reference.

The yeast and mold count was evaluated in all of the samples. The results of the experiments are shown in Table 3.

TABLE 3
Novel versus Conventional Sterilization Process

| Sample | | General | | | Average | % From |
|---|---|---|---|---|---|---|
| No. | Treatment | Count | Yeast | mold | Total Count | Control |
| 1, 2 | blender- processing (contyrol) | 170,000 | 15,000 | <10 | 150,000 | 100% |
| | | 130,000 | 7,800 | <10 | | |
| 3, 4 | blender- processing, & sterilizing | 1000 | <10 | <10 | 700 | 0.5% |
| | with hot water for 2 minutes | 400 | <10 | <10 | | |
| 5, 6 | blender- processing & | 130,000 | 1,300 | <10 | 120,000 | 80% |
| | 3 cycles of freezing/defrosting | 110,000 | 1,400 | <10 | | |
| 7, 8 | Processing using Liquid Nitrogen | 48,000 | 40 | <10 | 44,500 | 29.6% |
| | | 41,000 | 60 | <10 | | |
| 9, 10 | Processing using Liquid Nitrogen | 120 | <10 | <10 | 130 | 0.08% |
| | & Sterilization period with | 140 | <10 | <10 | | |
| | hot water | | | | | |
| 11, 12 | Processing using Liquid Nitrogen | 6500 | <10 | <10 | 16,500 | 11% |
| | & 3 cycles of freezing/defrosting | 27,000 | <10 | <10 | | |
| 13, 14 | Processing using Liquid Nitrogen | 7,300 | <10 | <10 | 5,100 | 3.4% |
| | & 6 cycles of freezing/defrosting | 2,900 | <10 | <10 | | |

Referring to samples 1 and 2 in Table-3, after crushing the plants by blender at the conventional way, the average total yeasts count was 150,000. However, after crushing the plants according to the novel way (see samples 7 and 8); the average total count was 44,500 and no yeasts were found such that a 70.4 % decrease in the bacteria count, samples 7 and 8.

Referring to samples 11 and 12, in Table-3, the plants were frozen, crushed, and the liquids extracted there from undergone sterilization process that included three cycles of freezing/defrosting, after which the total count was 16,500 such that a 89% decrease, samples 11,12.

Referring to samples 13 and 14, in Table-3, the plants were frozen, crushed, and the liquids extracted underwent a sterilization process that included six cycles of freezing/defrosting, after which the total count was only 5,100 (96.6% decrease), with no yeasts.

The lowest count, i.e., 130 (see samples 9 and 10) was obtained when the plants were frozen (i.e., with liquid nitrogen) and crushed, and the liquids extracted there from were sterilized by exposure to short heat treatment. However, it is known that utilization of heat in the sterilization process damages the quality of the product, because heat treatment tends to destroy most of the useful ingredients contained therein.

The separation and sterilization processes, as disclosed in the present invention, have proved to have the following advantages:
1) utilization of liquid nitrogen to freeze the plant which is an important step in both the separation process and in the sterilization process, resulted in dramatically reduced oxidation of the resulting treated plant(s), presumably due to the replacement of oxygen environment by nitrogen environment; and,
2) in the freezing-based separation and sterilization processes, there was only minor evaporation of aromatic substances, and, therefore, much of the aroma was sustained.

Thus, the experimental results demonstrate that the novel crushing method can indeed profitably replace both the conventional crushing process, and the conventional sterilization process. By freezing the plants prior to the crushing stage, and by replacing heat sterilization by freezing treatment (i.e., ‘cold sterilization’), the vitality of the solid residues and liquids extracted there from, is essentially sustained.

1. A process for separating between liquids extracted from whole plants or plant parts and the solid plant residue, and performing concurrent sterilization of both substances comprising:
a) fast deep-freezing said whole plant(s) or parts thereof;
b) crushing the deep-frozen whole plant(s), or parts thereof, thereby obtaining a bioextract;
c) defrosting said bioextract mixture so as to allow drainage of the liquids from the defrosted bioextract mixture; and,
d) separating between the liquids and the solid substance of the defrosted bioextract mixture such that a sterile product is obtained.

2. The process according to claim 1, wherein the separated liquids are further filtered and sterilized, the sterilization comprising employment of one or more “freeze-defrost” cycles, each cycles including one fast deep-freeze of the liquid(s) followed by one fast defrost of the frozen liquid(s).

3. The process according to claim 1, wherein the separated solids substances are further homogenized by processing the solid substances to an average particle size of no more than about 0.6 micron, and sterilized by employment of one or more “freeze-defrost” cycles, each cycle including one fast deep-freeze of the liquid(s) followed by one fast defrost of the frozen liquid(s).

4. The process according to claims 2 or 3, wherein in each freeze-defrost cycle, the liquids/solids substances are kept frozen at a temperature within the range of about −130° C. to −197° C. for a period of about 10 seconds, after which the frozen liquids/solids are fast defrosted to a temperature within the range of about 1 5° C. to 20° C., by immersing them in hot liquid, whose initial temperature is between about 80° C. and 90° C.

5. The process according to claim 4, wherein in each cycle of the sterilization process, the liquids/solids substances are frozen by utilizing liquid nitrogen.

6. The process according to claim 1, wherein the sterilized liquids and solids substances are utilized as ingredients in cosmetic, food, beverage, nutraceutical and pharmaceutical compositions by mixing them with at least one intermediator;

7. The process according to claim 6, wherein the intermediator is an option.

8. The process according to claim 1 wherein
a) at least one of the plants belongs to the labiatae family;
b) at least one of the plants is selected from the group of: Lavandula, Melissa, Mint, Ocimum, Origanum, Preslia, Rosmarinus, Salvia, Thymus;
c) leaves and/or shoots of the plant(s) are processed together with, or separately from, roots thereof; and/or
d) the mixing stage is preceded by washing the solid residues with a solvent selected from water and organic solvents, and the washed solid residues are subjected to homogenization and/or dehydration by freezing.

9. The process according to claim 1 wherein the process is utilized for making cosmetic, food, beverage, nutraceutical or pharmaceutical compositions, by: removing the liquids from whole plants, or parts of plants, after separating between the liquids and solids substances thereof, and mixing the residual solids substances, in particular the cell wall, with at least one cosmetically or pharmaceutically acceptable carrier, diluent, solvent or extractant.

10. The process according to claim 1, wherein the separated liquids and/or solid substances are utilized for making a beverage, by mixing the liquids and/or solids with at least one intermediator.

11. (canceled)

12. The process according to claim 1, wherein the solids and liquids of plants, or of selected parts of plants are utilized as natural colorants, flavorings and/or aromatics

13. Cosmetic, food, nutraceutical or pharmaceutical composition, which comprises a natural mineral-rich plant material obtained by: deep-freezing whole plant(s) or parts thereof; crushing the frozen whole plant(s) or parts thereof to a powder with a particles size within the range of 0.5 to 2 micron; defrosting the crushed product so as to allow drainage of liquids from a resulting bioextract; separating said liquids and the solid substance(s) of the bioextract; and, mixing the solid substance(s) together with at least one acceptable carrier, diluent, solvent or extractant.

14. The composition according to claim 13, wherein at least one of the plants, utilized for making said composition, belongs to the labiatae family.

15. The composition according to claim 13, which includes at least one plant selected from Lavandula, Melissa, Mint, Ocimum, Origanum, Preslia, Rosmarinus, Salvia and Thymus.

16. The composition according to claim 13, to which chlorophyll is added for protecting a skin.

17. The composition according to claim 13, wherein the solid substance(s) are homogenized by processing the solid substances to an average particle size of no more than about 0.6 micron, and sterilized, by employment of one or more “freeze-defrost” cycles, each cycle including one fast deep-freeze of the liquid(s) followed by one fast defrost of the frozen liquid(s).

18. The composition according to claim 17, wherein in each freeze-defrost cycle, the tissues are kept frozen in a temperature within the range of about −130° C. to −197° C. for a period of about 10 seconds, after which the frozen liquids/solids are fast defrosted to a temperature within the range of about 15° C. to −20° C., by immersing in hot liquid, having an initial temperature between about 80° C. and 90° C.

19. The composition according to any of claims 17 to 18, wherein in each cycle of the sterilization process, the tissues are frozen by utilizing liquid nitrogen.

20. Composition according to claim 13, wherein the materials are utilized as natural colorants, flavorings and/or aromatics.
